(19) United States
(12) Patent Application Publication
(10) Pub. No.: US 2020/0335193 A1
(43) Pub. Date: Oct. 22, 2020

(54) Pill Organization Utilizing A Voice-Based Virtual Assistant

(71) Applicant: Intellimed AI, Irvine, CA (US)

(72) Inventor: Amir Zaker, Tustin, CA (US)

(73) Assignee: Intellimed AI, Irvine, CA (US)

(21) Appl. No.: 16/786,261

(22) Filed: Feb. 10, 2020

Related U.S. Application Data
(60) Provisional application No. 62/837,170, filed on Apr. 22, 2019.

(51) Int. Cl. G16H 20/13; H04R 1/40; H04R 3/00; G10L 15/26; G10L 15/22; G10L 13/04; A61J 7/04; A61J 7/00; G16H 70/40; G16H 40/67; G16H 40/20; G16H 50/30; G16H 80/00; G16H 10/60; G06Q 10/10; G06Q 50/26; G06Q 10/08; G06K 7/14

(52) U.S. Cl. CPC G16H 20/13; H04W 4/80; H04R 3/005; G10L 15/265; G10L 15/22; G10L 13/043; A61J 7/0481; A61J 7/0007; A61J 7/0436; A61J 7/0076; G16H 70/40; G16H 40/67; G16H 40/20; G16H 50/30; G16H 80/00; G16H 10/60; G06Q 10/109; G06Q 50/265; G06Q 10/087; G06K 7/1417; G06K 7/1413; G10L 2015/223; H04R 1/406

(57) ABSTRACT

A system, device and method to assist users with taking their prescription medications, vitamins, insulin (alert only) and over the counter drugs are disclosed herein. The present invention assists the user with (including but not limited to): organizing their pills, taking their medication on time, taking the proper dosage of medication, providing warnings to avoid multiple/duplicative dosages, provide reminders to the user to take their medication on time, or remind them if they forgot to take their medication, and provide information regarding side effects of the medication and alert the user to dangers/risks of taking conflicting medications. The device preferably dispenses medication automatically based on pre-defined scripts and algorithms.

### CROSS REFERENCE TO RELATED APPLICATION

The Present application claims priority to U.S. Provisional Patent Application No. 62/837,170, filed on Apr. 22, 2019, which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The present invention generally relates to pill containers.

### Description of the Related Art

Consumers often forget to take their medications as well as those who do not know the proper dosages/instructions on how or when to take their medication. Elderly, blind/disabled, diabetics, supplement takers or those suffering from Alzheimer's have added obstacles to taking the proper dosage of medication on a regular basis.

There have been many attempts to improve medication adherence. For example, GlowCaps® from Vitality, Inc., Los Angeles, Calif. is a cap that screws-on to most standard, prescription medicine bottles. When a medication becomes due, both the cap and a plug-in nightlight flash orange to alert the patient of their dose. The cap transmits data to the plug-in device which ultimately communicates data over a cellular network. Patients receive a series of reminders after their medication is due, culminating in a reminder phone call to the patient or caregiver if the pill bottle is not opened. In addition to monitoring compliance, the GlowCaps® cap can also send a request for a new prescription via a push button that lies under the cap. Patients receive a call shortly after pushing the button, confirming their prescription.

MedPod, from Daya Medicals, Inc., Miami, Fla. (“DayaMed”), is a rechargeable and portable dispenser of pre-sorted medication. The MedPod reminds patients and/or their caregivers of missed doses via text, email, or by phone. LED lighting and audible reminders alert patients that they need to take their dose. DayaMed produces self-contained cartridges that can be loaded directly into the Medpod. These cartridges are shipped directly to patients, eliminating the need to manually load the device. Medpod is also equipped with a calling functionality that allows patients to directly contact their pharmacist or provider.

U.S. Pat. Nos. 8,744,620, 8,193,918, and U.S. Patent Publication Number 2013/0002795, assigned to MedMinder Systems, Inc., describe a technology enabled pill box that separates a patient's medications into 4 compartments for each day of the week. The compartments contain cups which in turn contain pills. Manipulating a lid, and/or placing into, removing from, or replacing a cup in a correct compartment are detected and compared to a medication dispensing compliance schedule. If a lid is not opened or a cup is not removed within a designated time frame, the patient receives an auditory prompt in addition to subsequent phone calls, text messages, and emails. The pill box can be equipped with wireless pendant that serves as a medical alert that connects patients to a medical professional at a certified monitoring center. MedMinder sells two basic types of pill boxes, one whose compartments remain locked until a dose is due and another whose compartments remain unlocked. Additionally, a patient can opt for MedMinder to issue trays, which can be filled by a caretaker to ease the burden of loading the pill box. The compartments of this tray contain the pill cups that the box would otherwise use.

U.S. Pat. No. 8,754,769, U.S. Patent Publication Number 2014/0347175, U.S. Patent Publication Number 2014/024094, and U.S. Patent Publication Number 2013/0222135, assigned to AdhereTech, Inc., describe smart pill bottles that track the exact amount of medication inside the bottle in real-time, wirelessly sends the data into the cloud, and reminds patients to take their dose via automated call or text message. In addition, the pill bottle is equipped with LED lighting to alert patients directly that a medication dose is due.

Other prior art solutions include Hero, Pillo, MED-Q, Forgetting the pill, Vaica, Livi Pill Dispenser, Timer Cap medicine bottle, MEDCENTER, GMS Group Medical Supply, LLC, lifeline philips, and Pillrite, information on each is available at the their respective website.

General definitions for terms utilized in the pertinent art are set forth below.

APP is a software application for a mobile phone such as a smart phone.

Application Programming Interface (API) is a collection of computer software code, usually a set of class definitions, that can perform a set of related complex tasks, but has a limited set of controls that may be manipulated by other software-code entities. The set of controls is deliberately limited for the sake of clarity and ease of use, so that programmers do not have to work with the detail contained within the given API itself.

BLUETOOTH technology is a standard short range radio link that operates in the unlicensed 2.4 gigaHertz band.

BLUETOOTH LOW ENERGY (aka “BLE” or “BLUETOOTH LE”) is a communication format from the Bluetooth Special Interest Group which uses the 2.4 gigaHertz band of BLUETOOTH technology but with a simpler modulation system that supports data pockets ranging from 8 to 27 octets, which are transferred at 1 Mbps.

FTP or File Transfer Protocol is a protocol for moving files over the Internet from one computer to another.

Hypertext Transfer Protocol (“HTTP”) is a set of conventions for controlling the transfer of information via the Internet from a web server computer to a client computer, and also from a client computer to a web server, and Hypertext Transfer Protocol Secure (“HTTPS”) is a communications protocol for secure communication via a network from a web server computer to a client computer, and also from a client computer to a web server by at a minimum verifying the authenticity of a web site.

Internet is the worldwide, decentralized totality of server computers and data-transmission paths which can supply information to a connected and browser-equipped client computer, and can receive and forward information entered from the client computer.

Media Access Control (MAC) Address is a unique identifier assigned to the network interface by the manufacturer.

SSID (Service Set Identifier) is a 1 to 32 byte string that uniquely names a wireless local area network.

Transfer Control Protocol/Internet Protocol (“TCP/IP”) is a protocol for moving files over the Internet.

URL or Uniform Resource Locator is an address on the World Wide Web.

User Interface or UI is the junction between a user and a computer program. An interface is a set of commands or menus through which a user communicates with a program. A command driven interface is one in which the user enter commands. A menu-driven interface is one in which the user selects command choices from various menus displayed on the screen.

Web-Browser is a complex software program, resident in a client computer, that is capable of loading and displaying text and images and exhibiting behaviors as encoded in HTML (HyperText Markup Language) from the Internet, and also from the client computer's memory. Major browsers include MICROSOFT INTERNET EXPLORER, NETSCAPE, APPLE SAFARI, MOZILLA FIREFOX, and OPERA.

Web-Server is a computer able to simultaneously manage many Internet information-exchange processes at the same time. Normally, server computers are more powerful than client computers, and are administratively and/or geographically centralized. An interactive-form information-collection process generally is controlled from a server computer, to which the sponsor of the process has access.

Wireless Application Protocol (“WAP”) is an open, global specification that empowers users with mobile wireless communication devices (such as mobile phones) to easily access data and to interact with Websites over the Internet through such mobile wireless communication device. WAP works with most wireless communication networks such as CDPD, CDMA, GSM, PDC, PHS, TDMA, FLEX, reflex, iDEN, TETRA, DECT, DataTAC, Mobitex and GRPS. WAP can be built on most operating systems including PalmOS, WINDOWS, CE, FLEXOS, OS/9, JavaOS and others.

Wireless AP (access point) is a node on the wireless local area network (WLAN) that allows wireless devices to connect to a wired network using Wi-Fi, or related standards.

There is still a need for assisting a user with taking medication on a regular basis and in the proper dosage.

### BRIEF SUMMARY OF THE INVENTION

A primary purpose of the present invention is to assist users with taking their prescription medications, vitamins, insulin (alert only) and over the counter drugs. The present invention assists the user with (including but not limited to): organizing their pills, taking their medication on time, taking the proper dosage of medication, providing warnings to avoid multiple/duplicative dosages, provide reminders to the user to take their medication on time, or remind them if they forgot to take their medication, and provide information regarding side effects of the medication and alert the user to dangers/risks of taking conflicting medications. The device preferably dispenses medication automatically based on pre-defined scripts and algorithms. The device may also assist in the process of getting prescription medication refills. The device will provide communication with the prescribing physician regarding refill assistance. The device can also calculate how many pills a user should take with them while travelling depending on duration of their trip. The device can also remind the user of their proper dosage and instructions on how to take the medication i.e. to take with a meal; full glass of water, on empty stomach etc. provided the dosage and regimen database. The device and its features can be used with voice activation and/or an application on the users' mobile device. The device will also have an emergency 911 button. Additional users and/or contacts, such as family members and caretakers, can register to be notified if the main user is not taking their medication. Users can personalize their device with a personalized name of their choice. The device can be programmed for use in multiple languages.

One aspect of the present invention is a system for pill organization utilizing a voice-based virtual assistant. The system comprises a pill container comprising a plurality of pill cells, a microphone array, a speech recognition software for converting speech to text and interpreting audio inputs, a database comprising data on medicines, a schedule for pill usage, a processor, a speaker for communicating with a user, a LCD panel for touch input, and a scanner. The microphone array collects audio inputs for interpretation by the speech recognition software. The processor processes speech and provides a response utilizing the speech recognition software. The scanner scans code labels on medicine packages. The system uses a voice-based virtual assistant to assist a user in taking pills according to the schedule for pill usage.

Another aspect of the present invention is a device for pill organization utilizing a voice-based virtual assistant. The device comprises a pill container comprising a plurality of pill cells, a microphone array, a speech recognition software for converting speech to text and interpreting audio inputs, a database comprising data on medicines, a schedule for pill usage, a processor, a speaker for communicating with a user, a LCD panel for touch input, and a scanner. The microphone array collects audio inputs for interpretation by the speech recognition software. The processor processes speech and provides a response utilizing the speech recognition software. The scanner scans code labels on medicine packages. The device uses a voice-based virtual assistant to assist a user in taking pills according to the schedule for pill usage.

Yet another aspect of the present invention is a non-transitory computer-readable medium that stores a program that causes a processor to perform functions for pill organization utilizing a voice-based virtual assistant by executing the following steps. Step one is receiving inputs regarding a pill usage for a user. Step two is receiving audio commands for the user. Step three is storing the inputs and audio commands in a database including a medication schedule. Step four is activating a pill container based on a medication schedule. Step five is opening a lid of a cell of the pill container according to the medication schedule. Step six is generating reminders to the user if the medication schedule is not followed.

Yet another aspect of the present invention is a system for pill organization utilizing a voice-based virtual assistant. The system comprises a device, a mobile application running on a mobile communication device, and a database comprising data on medicines, a schedule for pill usage. The device comprises a housing, a pill container comprising a plurality of pill cells, the pill container within the housing, a microphone array within the housing, a processor within the housing, a memory within the housing and in electrical communication with the processor, and comprising a speech recognition software for converting speech to text and interpreting audio inputs, a wireless transceiver in electrical communication with the processor and within the housing, a speaker for communicating with a user, a LCD panel on an exterior surface of the housing for touch input, and a scanner on an exterior of the housing. The microphone array collects audio inputs for interpretation by the speech recognition software. The processor processes speech and provides a response utilizing the speech recognition software. The scanner scans code labels on medicine packages. The system uses a voice-based virtual assistant to assist a user in taking pills according to the schedule for pill usage. The mobile application is in communication with the device and in communication with a pharmacy or physician.

Yet another aspect of the present invention is a device for pill organization utilizing a voice-based virtual assistant. The device comprises a pill container comprising a plurality of pill cells, a processor, and a voice-based virtual assistant mechanism for audibly communicating with a user. The voice-based virtual assistant mechanism collects audio inputs for interpretation by the speech recognition software. The processor processes speech and provides a response utilizing the voice-based virtual assistant mechanism. The device uses a voice-based virtual assistant to assist a user in taking pills according to the schedule for pill usage.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a flow chart of a general method for pill organization utilizing a voice-based virtual assistant.

FIG. 2 is a block diagram of a system for pill organization utilizing a voice-based virtual assistant.

FIG. 3 is a flow chart of a general method for medicine organization utilizing a voice-based virtual assistant.

FIG. 4 is a block diagram for medicine organization utilizing a voice-based virtual assistant.

### DETAILED DESCRIPTION OF THE INVENTION

Preferably, a user first manually inputs data regarding his/her prescriptions/medication into a database through a mobile app or uses the scanner on a device to scan the prescriptions to detect the prescription information. The system uses a speech recognition technology engine particularly tailored for the elderly, disabled, ill, travelers, and foreign language users and is trained on medical terminologies and other related voice based commands. The system stores this information in a database and proceses it to respond to the user. Once activated, the command/speech is converted into text, the text is analyzed and the meaning of the text is processed. The device uses its database (the database includes content domains, a calendar, prescription regimens, medicine information, and Command lists), to convert text into speech and communicate the results to the user. The LED indicator on the device then automatically activates and automatically opens a lid of the applicable pill container depending on the command. The device also self-activates based on the information input into the database. For example, the device self-activates to remind a user to take their medication at a certain time based on information in the database calendar. The device uses container sensors to know if and when a user has accessed a certain medication.

Software and hardware components of the invention include: Embedded Voice recognition SDK; Specific commands and natural language understanding; Bluetooth; Microphone; power source (Battery and plug-in); pill enclosure (with optional motorized components for automatic dispensing); LED indicator; CPU; Speaker; Barcode reader/scanner; LCD panel—to get input through touch; Optional wifi; and a Pill Cuter.

The Present Invention assists users in taking their medications, vitamins, and prescriptions on time and properly: including instruction on how to take the medication, for example, “Take with a full glass of water.”

The Present Invention organizes medication; provides warnings and reminders regarding dosage, side effects, risks, refills etc.; and provides information regarding usage of medication during travel.

The Present Invention also provides automated dispensing of medications; voice activation solves the issue of technology or language barriers; provides emergency assistance; and allows for family and caretakers to be updated and informed on users' activity.

Information may be inputted into the system by any another person if the user is not capable of doing so.

The Present Invention allows a user to follow a regimen and dosage of medications for on-time and proper administration.

The Present Invention allows a user to avoid forgetting medications.

The Present Invention allows a user to manage medicine when traveling.

The Present Invention provides a user a refill alert.

The Present Invention includes voice activated hardware product that understand elderly and medical terminologies to dispense the right medication.

The Present Invention monitors consumption and direction of medication.

The Present Invention provides a connection with a physician for a refill.

The Present Invention provides a connection with a physician for inquiries that are not supported by the device.

The Present Invention provides a dashboard of medicine used with the correct direction and regimen.

The Present Invention provides an input on medicine conflicts, side-effects, etc.

The present invention provides interactive guidance to the patient to fill up the box based on instructions on the prescription. The present invention interacts with the user through box or app on any possible medicine conflicts. The present invention notifies the patient through app or box of any timing issues (taking meds, missed meds, upcoming meds). The device and/or app provides direction to the user on how to take the medicines. The device and/or app alerts the patient/user of side effects/adverse effects notices. The device and/or app provides suggestions on progression of taking medicines. The device and/or app schedules doctor appointments through telemedicine or other means of communicating with a physician's office. The device and/or app alerts the patient to order medicine refills from a pharmacy. The device also connects to a pharmacy on file with the device or app. The device makes a telecommunication or video connection with the user's physician or pharmacist. The device through voice can call 911 in an emergency.

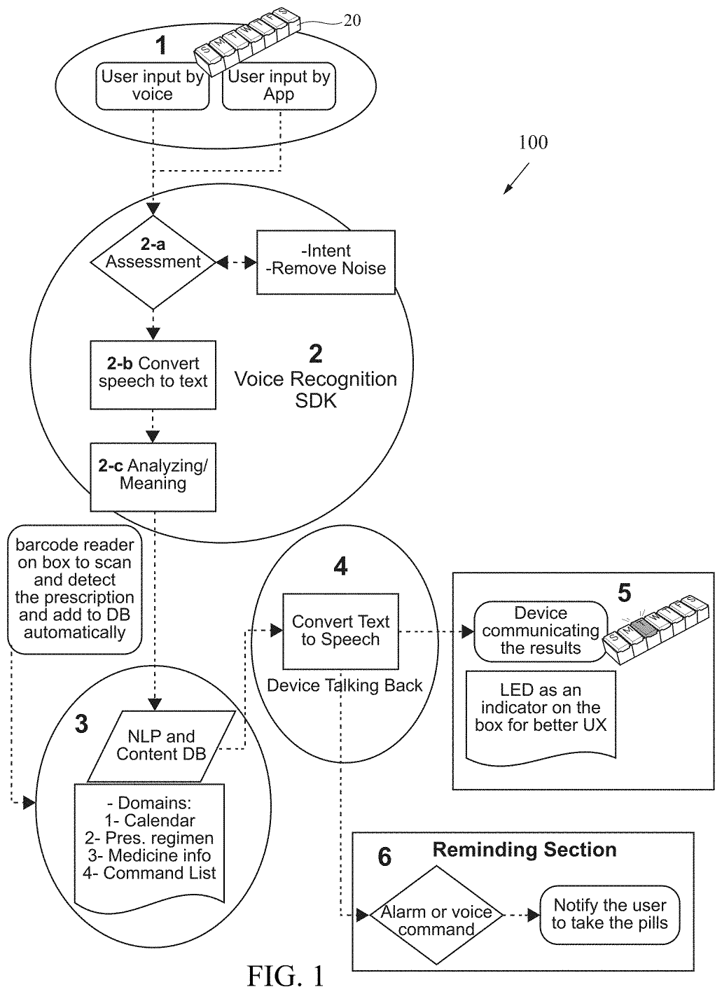

FIG. 1 is a flow chart of a general method for pill organization utilizing a voice-based virtual assistant. As shown in FIG. 1, a stage 1, a user inputs prescription information by voice or using a mobile app on a mobile communication device into a pill container 20. At stage 2, voice recognition software receives the voice input from the user. At sub-stage 2-a, the voice recognition software assesses voice information. At sub-stage 2-b, the voice recognition software converts the speech to text. At sub-stage 2-c, the voice recognition software performs an analysis on the meaning of the text. A barcode reader/scanner on the pill container scans and detects a prescription and automatically adds the information to a prescription database. At stage 3, the analysis is transmitted to a content database, which contains a calendar, a prescription medicine regimen, information of medicines used by the user, a command list, and the like. At stage 4, text is converted to speech for the device to provide audio alerts and prescription medicine information to the user. At stage 5, the device communicates with the user, and also uses a LED as an indicator on the device to provide user information to the user. At stage 6, a reminder stage, the device provides alarms, either audio, visual and/or tactile, to remind the user to take a prescribed medicine.

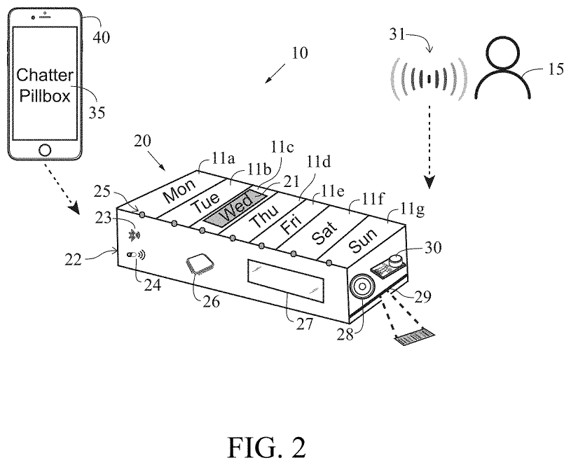

One embodiment is a system for pill organization utilizing a voice-based virtual assistant. As shown in FIG. 2, the system comprises a pill container 20 comprising pill cells 11a-11g, a microphone array 30 with a speech recognition software for converting speech to text and interpreting audio inputs, a database 32 comprising data on medicines, a schedule for pill usage, a processor 26, a speaker 28 for communicating with a user, a LCD panel 27 for touch input, a WiFi transceiver 24, a BLUETOOTH transceiver 23, and a scanner 29. The microphone array 30 collects audio inputs (voice requests 31) from a user 15 for interpretation by the speech recognition software. The processor 27 processes speech and provides a response utilizing the speech recognition software. The scanner 29 scans code labels on medicine packages. The system 10 uses a voice-based virtual assistant to assist a user 15 in taking pills according to the schedule for pill usage.

The system 10 further comprises a power source for powering the system, a pill cutter 22 for cutting pills, and a transceiver 24 for wireless communications.

The system further comprises a lid sensor 25 on each lid of each pill cell of the pill cells 11a-11g for the pill container 20, wherein the lid sensor 25 registers activity with the database 32 when opened.

The system 10 further comprises a mobile application 35 for a mobile communication device 40, the mobile application 35 registering the user, activating components, providing updates and other communications to the user 15.

The system 10 further comprises a motorized component for each lid of each pill cell of the plurality of pill cells 11a-11g for the pill container 20, wherein the motorized component opens and closes the lid automatically.

The system 10 further comprises a charging nest for charging a battery.

The pill container 20 further comprises a LED indicator 21 for using light to communicate with a user.

The pill container 20 is preferably filled with at least one of prescription medicine, vitamin, insulin, and over-the-counter drugs.

Another embodiment is a device 20 for pill organization utilizing a voice-based virtual assistant. The device 10 comprises a plurality of pill cells 11a-g, a microphone array 30, a speech recognition software for converting speech to text and interpreting audio inputs, a database 32 comprising data on medicines, a schedule for pill usage, a processor 26, a speaker 28 for communicating with a user, a LCD panel 27 for touch input, and a scanner 29. The microphone array collects audio inputs for interpretation by the speech recognition software. The processor processes speech and a provides a response utilizing the speech recognition software. The scanner scans code labels on medicine packages. The device uses a voice-based virtual assistant to assist a user in taking pills according to the schedule for pill usage.

Yet another embodiment is a device for pill organization utilizing a voice-based virtual assistant. The device comprises a pill container comprising a plurality of pill cells, a processor, and a voice-based virtual assistant mechanism for audibly communicating with a user. The voice-based virtual assistant mechanism collects audio inputs for interpretation by the speech recognition software. The processor processes speech and provides a response utilizing the voice-based virtual assistant mechanism. The device uses a voice-based virtual assistant to assist a user in taking pills according to the schedule for pill usage.

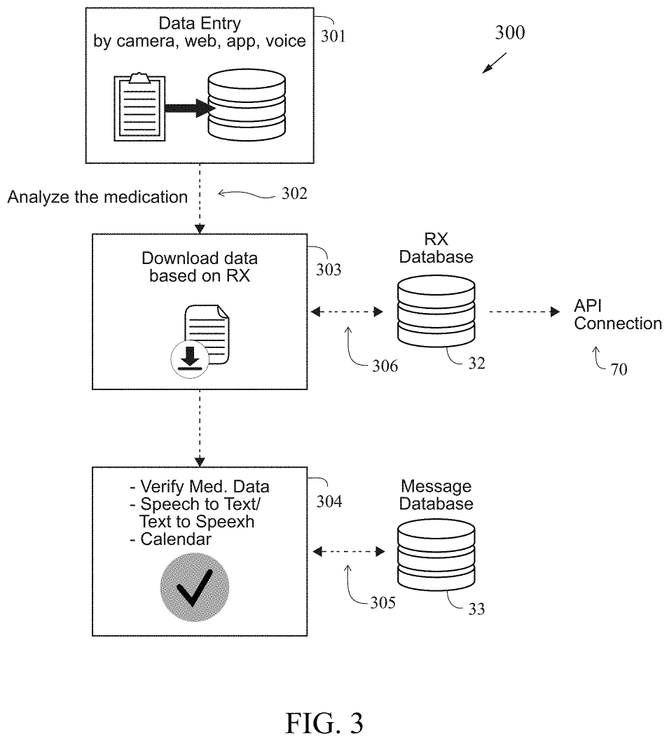

FIG. 3 is a flow chart of a general method 300 for medicine organization utilizing a voice-based virtual assistant. As shown in FIG. 3, a block 301, prescription data is entered into a database. At transition 302, the medication is analyzed. At block 303, data for the prescribed medication is downloaded at transition 304 from a prescription database 32 which has an API connection to online sources for information on prescribed medicines. At block 304, medicine data is verified on a device, and a message database 33 provides voice information at transition 305 to a device.

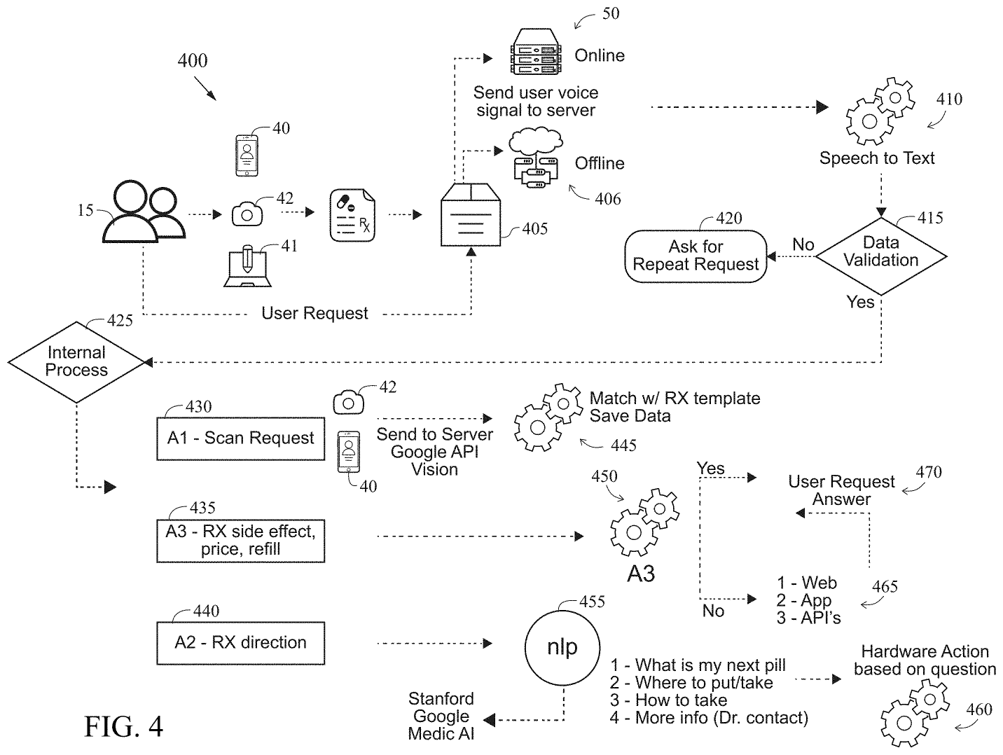

FIG. 4 is a block diagram 400 for the mechanism for medicine organization utilizing a voice-based virtual assistant. A user 15 first manually inputs data regarding his/her prescriptions/medication into the system through a mobile app on a mobile phone 40, a camera 42 or a computer (laptop or tablet) 41. At 405, the data is sent to a server 50 if online, or cached at 406 if offline. At 410, the speech is converted to text and at step 415 is the data is validated at the server. If the data cannot be validated, at step 420 a message is sent to the user to repeat the request. If the data is validated, then at step 425 the internal process begins with a scan request 430 of a barcode (or QR code) for a prescription, prescription side effects, prices, refills at step 435, and prescription directions at step 440. The scan request 435 is perform by a camera 42 or mobile app on a mobile device 40. The data is sent to the server using an API to match the prescription template and save the data at step 445. At step 450, the server determines if the prescription data (side effects, price, refills, etc.) is provided and if not, the server searches the web, applications, API's for the data or requests response from the user. At step 455, the nlp (neuro-linguistic programming) engine using the voice-based virtual assistant to provide directions to the user. At step 460, hardware action is taken based on a question from the user.

An operating system controls the execution of other computer programs, running of the PSO platform, and provides scheduling, input-output control, file and data management, memory management, and communication control and related services. The operating system may be, for example Windows (available from Microsoft, Corp. of Redmond, Wash.), LINUX or other UNIX variants (available from Red Hat of Raleigh, N.C. and various other vendors), Android and variants thereof (available from Google, Inc. of Mountain View, Calif.), Apple OS X, iOs and variants thereof (available from Apple, Inc. of Cupertino, Calif.), or the like.

The method described in connection with the embodiments disclosed herein is preferably embodied directly in hardware, in a software module executed by a processor, or in a combination of the two. A software module preferably resides in flash memory, ROM memory, EPROM memory, EEPROM memory, RAM memory, registers, a hard disk, a removable disk, a CD-ROM, or any other form of storage medium known in the art. An exemplary storage medium is preferably coupled to the processor, so that the processor reads information from, and writes information to, the storage medium. In the alternative, the storage medium is integral to the processor. In additional embodiments, the processor and the storage medium reside in an Application Specific Integrated Circuit (ASIC). In additional embodiments, the processor and the storage medium reside as discrete components in a computing device. In additional embodiments, the events and/or actions of a method reside as one or any combination or set of codes and/or instructions on a machine-readable medium and/or computer-readable medium, which are incorporated into a computer software program.

In additional embodiments, the functions described are implemented in hardware, software, firmware, or any combination thereof. If implemented in software, the functions are stored or transmitted as one or more instructions or code on a computer-readable medium. Computer-readable media includes both computer storage media and communication media including any medium that facilitates transfer of a computer program from one place to another. A storage medium is any available media that is accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures, and that can be accessed by a computer. Also, any connection is termed a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc”, as used herein, include compact disc (CD), laser disc, optical disc, digital versatile disc (DVD), floppy disk and BLU-RAY disc where disks usually reproduce data magnetically, while discs usually reproduce data optically with lasers. Combinations of the above should also be included within the scope of computer-readable medium.

A computer program code for carrying out operations of the Present Invention is preferably written in an object oriented, scripted or unscripted programming language such as C++, C #, SQL, Java, Python, Javascript, Typescript, PHP, Ruby, or the like.

Each of the interface descriptions preferably discloses use of at least one communication protocol to establish handshaking or bi-directional communications. These protocols preferably include but are not limited to XML, HTTP, TCP/IP, Serial, UDP, FTP, Web Services, WAP, SMTP, SMPP, DTS, Stored Procedures, Import/Export, Global Positioning Triangulation, IM, SMS, MMS, GPRS and Flash. The databases used with the system preferably include but are not limited to MS SQL, Access, My SQL, Oracle, DB2, Open Source DBs and others. Operating system used with the system preferably include Microsoft 2010, XP, Vista, 200o Server, 2003 Server, 2008 Server, Windows Mobile, Linux, Android, Unix, I series, AS 400 and Apple OS.

The underlying protocol at a server, is preferably Internet Protocol Suite (Transfer Control Protocol/Internet Protocol (“TCP/IP”)), and the transmission protocol to receive a file is preferably a file transfer protocol (“FTP”), Hypertext Transfer Protocol (“HTTP”), Secure Hypertext Transfer Protocol (“HTTPS”), or other similar protocols. The protocol at the server is preferably HTTPS.

Components of a server includes a CPU component, a graphics component, memory, non-removable storage, removable storage, Network Interface, including one or more connections to a fixed network, and SQL database(s). Included in the memory, is an operating system, a SQL server or other database engine, and computer programs/software.

A mobile communication devices utilized with the present invention preferably include mobile phones, smartphones, tablet computers, PDAs and the like. Examples of smartphones include the IPHONE® smartphone from Apple, Inc., BLACKBERRY® smartphones from Research In Motion, the DROID® smartphone from Motorola Mobility Inc., GALAXY S® smartphones from Samsung Electronics Co., Ltd, and many more. Examples of tablet computing devices include the IPAD® tablet from Apple Inc., and the GALAXY TAB® tablet from Samsung Electronics Co., Ltd.

A typical mobile communication device includes an accelerometer, I/O (input/output), a microphone, a speaker, a GPS chipset, a Bluetooth component, a Wi-Fi component, a 3G/4G component, RAM memory, a main processor, an OS (operating system), applications/software, a Flash memory, SIM card, LCD display, a camera, a power management circuit, a battery or power source, a magnetometer, and a gyroscope.

A mobile communication service provider (aka phone carrier) of the customer such as VERIZON, AT&T, SPRINT, T-MOBILE, and the like mobile communication service providers, provide the communication network for communication to the mobile communication device of the end user.

Wireless standards include 802.11a, 802.11b, 802.11g, AX.25, 3G, CDPD, CDMA, GSM, GPRS, radio, microwave, laser, Bluetooth, 802.15, 802.16, and IrDA.

Components of a server includes a CPU component, a graphics component, PCI/PCI Express, memory, non-removable storage, removable storage, Network Interface, including one or more connections to a fixed network, and SQL database(s), which includes the venue's CRM. Included in the memory, is an operating system, a SQL server or other database engine, and computer programs/software. The server also includes at least one computer program configured to receive data uploads and store the data uploads in the SQL database. Alternatively, the SQL server can be installed in a separate server from the venue server.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

1. A system for pill organization utilizing a voice-based virtual assistant, the system comprising:
a device comprising
a pill container comprising a plurality of pill cells,
a microphone array,
a speech recognition software for converting speech to text and interpreting audio inputs,
a database comprising data on medicines, a schedule for pill usage,
a processor,
a voice-based virtual assistant for communicating with a user, and
a scanner;
wherein the microphone array is configured to collect a plurality of audio inputs for interpretation by the speech recognition software;
wherein the processor processes speech and a provides a response utilizing the speech recognition software, wherein the speech recognition software is configured to receive a voice input from a user, wherein the speech recognition software is configured to assess the voice input from the user for an intent of the user, wherein the speech recognition software is configured to convert speech from the voice input to text, wherein the speech recognition software is configured to perform an analysis on the meaning of the text;
wherein the scanner scans code labels on medicine packages and the processor analyzes the data from the code labels with information stored in the database to detect for a medication conflict;
wherein the device uses the voice-based virtual assistant to assist a user in taking pills according to the schedule for pill usage.

2. The system according to claim 1 further comprising a server for communicating medication data to the device.

3. The system according to claim 1 further comprising a pill cutter for cutting pills.

4. The system according to claim 1 further comprising a transceiver for wireless communications.

5. The system according to claim 1 further comprising a lid sensor on each lid of each pill cell of the plurality of pill cells for the pill container, wherein the lid sensor registers activity with the database when opened.

6. The system according to claim 1 further comprising a mobile application for a mobile communication device, the mobile application registering the user, activating components, providing a plurality of updates and a plurality of other communications to the user.

7. The system according to claim 1 further comprising a motorized component for each lid of each pill cell of the plurality of pill cells for the pill container, wherein the motorized component opens and closes the lid automatically.

8. The system according to claim 6 wherein the device or the mobile application is configured to alert the user of at least one of adverse effects of a medication, a need to order refills of a medication from a pharmacy, a timing issue for taking a medication, a timing issue for failure to take a medication, a progression of taking a medication, or guidance of how to take a medication.

9. The system according to claim 1 wherein the device or the mobile application is configured to communicate with at least one of a physician's office to schedule a visit, a pharmacy to refill a prescription, or emergency services.

10. The system according to claim 1 wherein the processor is configured to organize a user's pills, to schedule medication, to provide the proper dosage of medication, provide warnings to avoid multiple/duplicative dosages, provide reminders to the user to take their medication on time, or remind them if they forgot to take their medication, and provide information regarding side effects of the medication and alert the user to dangers/risks of taking conflicting medications.

11. The system according to claim 1 wherein the processor is configured to assist in the process of getting prescription medication refills and communicating with a prescribing physician regarding refill assistance.

12. A method for pill organization utilizing a voice-based virtual assistant, the method comprising:
receiving prescription data for a prescribed medication for a user;
receiving medication data for the medication through an application programming interface (API) connection;
retrieving current medication data from a database of a device containing a list of current medication used by the user;
analyzing the prescription data, the medication data and the current medication data to detect a conflict with the prescribed medication and the current medication of the user;
communicating the conflict detection analysis to the user; and
adding the prescribed medication to the database for the device and establishing a schedule for reminding the user to take the prescribed medication;
wherein the device comprises
a pill container comprising a plurality of pill cells,
a microphone array,
a speech recognition software for converting speech to text and interpreting audio inputs,
the database comprising data on medicines, a schedule for pill usage,
a processor,
a voice-based virtual assistant for communicating with a user, and
a scanner;
wherein the speech recognition software is configured to receive a voice input from a user, wherein the speech recognition software is configured to assess the voice input from the user for an intent of the user, wherein the speech recognition software is configured to convert speech from the voice input to text, wherein the speech recognition software is configured to perform an analysis on the meaning of the text.

13-15. (canceled)

16. A device for pill organization utilizing a voice-based virtual assistant, the device comprising:
a pill container comprising a plurality of pill cells;
a processor; and
a voice-based virtual assistant mechanism for audibly communicating with a user;
wherein the voice-based virtual assistant mechanism collects audio inputs for interpretation by the speech recognition software, wherein the speech recognition software is configured to receive a voice input from a user, wherein the speech recognition software is configured to assess the voice input from the user for an intent of the user, wherein the speech recognition software is configured to convert speech from the voice input to text, wherein the speech recognition software is configured to perform an analysis on the meaning of the text;
wherein the processor processes speech and a provides a response utilizing the voice-based virtual assistant mechanism;
wherein the device uses a voice-based virtual assistant to assist a user in taking pills according to the schedule for pill usage.

17. The device according to claim 16 wherein the voice-based virtual assistant comprises a microphone array, a speaker and a speech recognition software for converting speech to text and interpreting audio inputs.

18. The device according to claim 16 wherein the processor is configured to organize a user's pills, to schedule medication, to provide the proper dosage of medication, provide warnings to avoid multiple/duplicative dosages, provide reminders to the user to take their medication on time, or remind them if they forgot to take their medication, and provide information regarding side effects of the medication and alert the user to dangers/risks of taking conflicting medications.

19. The device according to claim 16 wherein the processor of the device is configured to alert the user of at least one of adverse effects of a medication, a need to order refills of a medication from a pharmacy, a timing issue for taking a medication, a timing issue for failure to take a medication, a progression of taking a medication, or guidance of how to take a medication.

20. The device according to claim 16 wherein the processor device is configured to communicate with at least one of a physician's office to schedule a visit, a pharmacy to refill a prescription, or emergency services.
